(19) United States
(12) Patent Application Publication
(10) Pub. No.: US 2008/0105318 A1
(43) Pub. Date: May 8, 2008

(54) Turbulence Minimizing Device for Multi-Lumen Fluid Infusing Systems and Method for Minimizing Turbulence in Such Systems

(76) Inventor: James E. Leone, Pinecrest, FL (US)

Correspondence Address: MAYBACK & HOFFMAN, P.A., 5722 S. FLAMINGO ROAD #232, FORT LAUDERDALE, FL, 33330, US

(21) Appl. No.: 11/870,227

(22) Filed: Oct. 10, 2007

Related U.S. Application Data
(60) Provisional application No. 60/851,035, filed on Oct. 11, 2006.

(51) Int. Cl. A61M 39/10

(52) U.S. Cl. 137/896

(57) ABSTRACT

A fluid turbulence minimizing device includes a connection portion defining a connection cavity shaped to receive a tertiary fluid-supply conduit including a primary fluid-supply lumen and two secondary fluid-supply lumens and an intermixing portion having a longitudinal flow axis and defining an intermixing cavity that has an input orifice fluidically communicating with the connection cavity and a given area, an exit orifice having an area less than the given area, an inner surface having an upstream side adjacent the connection portion, a downstream side at a distance from the connection portion, and a cross-sectional area decreasing from the input orifice to the exit orifice to convey fluid supplied from the conduit to the connection cavity through the intermixing cavity and out the exit orifice, and guide fins inwardly projecting from the inner surface of the intermixing portion toward the longitudinal flow axis and having a longitudinal extent aligned substantially parallel with the longitudinal flow axis.

### CROSS-REFERENCE TO RELATED APPLICATION

This application claims the priority, under 35 U.S.C. § 119, of U.S. Provisional Patent Application No. 60/851,035 filed Oct. 11, 2006, the entire disclosure of which is hereby incorporated herein by reference in its entirety.

### BACKGROUND OF THE INVENTION

1. Field of the Invention

The present invention relates to a turbulence minimizing connector for allowing multiple streams of liquid to enter the connector, flow together therein, and exit the connector with minimal mixing.

2. Description of the Prior Art

Heretofore, multiple fluid-carrying lumens (also referred to as extensions, catheters or multi-lumen catheters) have been proposed for mixing components therein prior to delivery of the mixture to a patient, i.e., a human body.

FIG. 1 depicts an intravenous extension system 10 described in U.S. Pat. No. 6,780,167 issued to the inventor of the instant application on Aug. 24, 2004 (the “'167 Patent” or “'167”). The '167 Patent includes a multi-lumen intravenous extension 12 having a connector 14 at the proximal end 16 thereof. Connected to the proximal end 16 of the extension 12 is a main infusion conduit or tubing 18. The tubing 18 is connected to an upstream connector 20 that, in turn, is fluidically connected to tubing 22 extending from a fluid source 24 (e.g., a bag of saline solution). Also connected to the connector 14 are first and second tubes 26, 27, which are connected to first and second fluid sources 28, 29, respectively (e.g., syringes). Each of the fluid sources 28, 29 can contain a selected drug, medication, or liquid in a predetermined amount that is to be infused into a body 28.

At a distal end 30 of the extension 12 is a coupling connector 32 that includes a mixing or “common” chamber 33 illustrated in FIGS. 2 to 4. Distal connectors existing prior to the '167 Patent were realized by an industry standard connector, referred to as a luer connector 32. The '167 Patent supplied the connector 32 to provide a fluidic interface between the extension 16 and an infusion needle or intravenous catheter 34. The outlet of the connector 32 is connected to the needle/catheter 34, which is inserted into the body 38 and is, typically, held therein by a wing tape or bandage 40.

FIGS. 2 to 4 illustrate that the mixing chamber 33 in the connector 32 has an outer cylindrical wall or tubular portion 42 that is received over the distal end 30 of the multi-lumen intravenous extension tubing 12.

FIG. 3, in particular, shows that the tubular portion 42 of the connector 32 has a larger diameter to fit over the distal end 30 of the extension tubing 12. The exit portion 44 at the distal end of the connector 32 fits over or is connected to a proximal end 46 of the catheter 34 or needle. As such, the connector 32 provides the mixing chamber 33 for liquids, including a main liquid such as a saline solution and one or more medications or other liquids provided through the tubes 26, 27. This mixing chamber 33 is smooth-bored throughout. The diameter abruptly changes between the tubular portion 42 and the exit portion 44 at an interface 35 (indicated by dashed lines in FIG. 3).

With multi-lumen medical tubing (e.g., the multi-lumen intravenous extension 12 of the '167 Patent), fluids exit the connector 14 (or the extension 16) along with the fluid passing through the main lumen 52. It would be beneficial to have these fluids not intermix and remain separated for as long as possible, prior to vascular entry. If such intermixing is prevented, then the intended additionally added medication is administered with a minimal degree of dilution and/or interaction with other medications until it enters the vessel intended to receive such medications. As such, unwanted boluses of medication and interactions are avoided. It is known that medications, especially, injected anesthesia, should be administered with constancy and control, and not with randomly sized or chaotic boluses because differential administration of such medicines can have serious, if not deadly, consequences.

Based upon the above considerations, it would be beneficial to provide a device that minimizes turbulence of the co-delivered fluids.

### SUMMARY OF THE INVENTION

It is accordingly an object of the invention to provide a turbulence minimizing device for multi-lumen fluid delivery systems and a method for minimizing turbulence in such systems that overcome the hereinafore mentioned disadvantages of the heretofore-known devices and methods of this general type and that are configured to integrate with existing standardized infusion systems to minimize chaotic admixing of fluids that are to be transfused concomitantly.

The present invention is an improvement upon prior art connectors for infusion systems. In one exemplary embodiment, the present invention improves upon the multi-lumen intravenous extension described in the '167 Patent. This extension is used for transmitting liquids in a body and for infusing the fluids individually undiluted and unprecipitated as close as possible to the point where they are injected into the blood stream, for example. While the turbulence minimizing device of the present invention can be used with the multi-lumen extension of the '167 Patent, it is not limited to use with this device. The present invention, however, is particularly useful when combined with the '167 device and, therefore, portions of the '167 disclosure are included herein. For clarity, the '167 disclosure is incorporated by reference herein in its entirety. Inclusion of the '167 catheter herein should not be taken as applicable only to this exemplary embodiment of a medical fluid infusing device. Those having ordinary skill in the art of such devices will appreciate the improvement that the present invention may provide to other prior art devices that deliver medicinal fluids.

The connector of the present invention is positioned between an intra-vascular or intravenous access site and an infusion system typically including a steady supply of saline and syringes or syringe connectors or medicinal fluid pumps predetermined for injecting amounts of drugs, medications, or other liquids. The connector of the present invention allows for organized and controllable delivery and administration of a wide variety of medications and pharmaceutical agents with a minimal amount of medication intermixing prior to entry into a body.

The mixing connector forms the male half of a luer lock connector. The mixing connector has a size equal to the medical industry standard for insertion into a vascular access device. The term “standard,” as it is used herein, relates to the industry standard corresponding to ISO 594-1:1986.

When used with the multi-lumen intravenous extension of the '167 Patent, the connector of the present invention replaces the connector 32, which is positioned between the intra-vascular or intravenous access site and the multi-lumen intravenous extension 12.

The Coanda Effect, also known as “boundary layer attachment”, is the tendency of a stream of fluid to stay attached to a convex surface, rather than follow a straight line in its original direction even if the surface's direction of curvature is directed away from the axis of the stream of fluid. The mixing connector of the invention utilizes the Coanda Effect when directing the stream of liquids exiting a multi-lumen interface (such as the distal end 30). In particular, the fluids exiting secondary lumens that are disposed adjacent the inner wall of the mixing connector will travel along that surface and remain substantially coherent along the convex surface with little or no mixing with the fluid exiting the primary lumen or other fluid(s) exiting secondary lumen(s). This laminar flow is maintained most or all of the way through the mixing connector. It can be appreciated that this laminar flow is enhanced when guiding fins project inwardly from the surface over which the fluids travel. The mixing connector contains features to take advantage of the Coanda Effect. In this way, different medications can be kept separate, independent of carrier flow rate and boluses. Because differing drugs are sometimes incompatible, e.g., due to differing drug solubilities that can cause undesirable precipitant or can cause drug inactivation, it is desirable to keep the drugs separate before introduction into a patient. Such separation is important to drugs like Dilantin/phenytoin, which precipitates when piggybacked into any dextrose-containing solution. The phenomenon relates, often, to the solute and the solvent (pH, concentration, temperature in solution, protein binding, etc.). Amphotericin B (Fungizone) similarly precipitates with solutions containing sodium chloride and Dopamine (Intropin, Revimine) is inactivated in solutions with a high pH and must not be piggybacked into any solution containing sodium bicarbonate. The mixing connector of the invention reduces the possibility of drug incompatibility to a point where mixture and common exposure is substantially eliminated and the possibility of drug precipitation is minimized.

The mixing connector can be used in a number of medical applications, such as with delivery of anesthesia during operations. The mixing connector allows for infusion of anesthetic agents, vaso-active agents, antibiotics, and antiarrhymics, whether in adults or children (both neonatal and pediatric) and can be used with a patient controlled analgesia (PCA) pump. Also, the mixing connector can be used in an intensive care unit for vaso-active medications, antiarrhymics, potassium, antibiotics, insulin, etc.

The '167 Patent describes an over-pressure danger that exists at a vascular entry point when fluid is being introduced into a patient. As can be understood from the description of the mixing connector herein, replacement of the '167 connector 32 with the mixing connector does not adversely impact the over-pressure protection that exists when the mixing connector is utilized with the '167 system 10 and, therefore, is particularly suited for improving that system 10.

Because turbulence of the intermixed fluids at the mixing connector is minimized, all of the advantages provided by the '167 system remain with the connector of the present invention. More specifically, delivering the pharmaceutical agents with less change to the normal fluid dynamics improves patient safety as compared to prior art infusion systems. Additionally, the infusion of liquid agents through the satellite lumens remains independent upon carrier fluid rates for delivery. Because the liquids from the satellite lumens are delivered with greater control in volume, time of onset of the action of the agents delivered is decreased and the concentration of those agents remains virtually constant. Less intermixing of the fluids also means that delivery of the agents infused through the satellite lumens will not be altered by the carrier fluid rate. Like the '167 system, the mixing connector decreases priming volume even more by further reducing the “tubing dead space.” The mixing connector also allows and enhances independent infusion of multiple agents and reduces carrier fluid rate requirements.

Other features that are considered as characteristic for the invention are set forth in the appended claims.

Although the invention is illustrated and described herein as embodied in a turbulence minimizing device for multi-lumen fluid infusing devices and a method for minimizing turbulence in such systems, it is, nevertheless, not intended to be limited to the details shown because various modifications and structural changes may be made therein without departing from the spirit of the invention and within the scope and range of equivalents of the claims.

The construction and method of operation of the invention, however, together with additional objects and advantages thereof, will be best understood from the following description of specific embodiments when read in connection with the accompanying drawing.

### BRIEF DESCRIPTION OF THE DRAWINGS

In the following, the invention will be described in more detail by exemplary embodiments and the corresponding figures. By schematic illustrations that are not true to scale, the figures show different exemplary embodiments of the invention. The same or equally functioning parts are characterized with the same reference numerals. Shown are sections in schematic cross-section.

FIG. 1 is a fragmentary, perspective and partially cut-away view of a prior art multi-lumen intravenous extension coupled at its input to a bag of saline solution and to two syringes and coupled at its output end to an infusion catheter inserted into a body;

FIG. 2 is a cross-sectional view of a mixing chamber connector of FIG. 1 along section line 2-2;

FIG. 3 is a cross-sectional view of the mixing chamber connector of FIG. 2 along section line 3-3;

FIG. 4 is a cross-sectional view of a mixing chamber connector according to the present invention viewed along section line 4-4 with a fluid supply input having a main lumen and two satellite lumens;

FIG. 5 is a fragmentary, cross-sectional view of a connector according to the invention coupled to a downstream female luer connector;

FIG. 6 is a cross-sectional view of a first alternative exemplary embodiment of a portion of the connector of FIG. 5 with two satellite lumens and a primary lumen with fins separating the openings of the two satellite lumens from one another;

FIG. 7 is a cross-sectional view of a second alternative exemplary embodiment of a portion of the connector of FIG. 5 with two satellite lumens and a primary lumen with fins separating the openings of the two satellite lumens from one another;

FIG. 8 is a cross-sectional view of a third alternative exemplary embodiment of a portion of the connector of FIG. 5 with three satellite lumens and a primary lumen with fins separating the openings of the three satellite lumens from one another;

FIG. 9 is a cross-sectional view of a fourth alternative exemplary embodiment of a portion of the connector of FIG. 5 with four satellite lumens and a primary lumen with fins separating the openings of the four satellite lumens from one another;

FIG. 10 is a cross-sectional view of a fifth alternative exemplary embodiment of a portion of the connector of FIG. 5 with four satellite lumens and a primary lumen with fins separating the openings of the four satellite lumens from one another;

FIG. 11 is a cross-sectional view of a sixth alternative exemplary embodiment of a portion of the connector of FIG. 5 with six satellite lumens and a primary lumen with fins separating the openings of the six satellite lumens from one another;

FIG. 12 is a cross-sectional view of a seventh alternative exemplary embodiment of a portion of the connector of FIG. 5 with two satellite lumens and a primary lumen with fins bisecting the openings of the satellite lumens;

FIG. 13 is a cross-sectional view of a eighth alternative exemplary embodiment of a portion of the connector of FIG. 5 with six satellite lumens and a primary lumen with fins bisecting the openings of the satellite lumens;

FIG. 14 is a fragmentary, cross-sectional view of an alternative embodiment of the connector according to the invention along section line 14-14 in FIG. 15 and coupled to a downstream female luer connector;

FIG. 15 is a cross-sectional view of the connector of FIG. 14 along section line 15-15 in FIG. 14;

FIG. 16 is a side elevational view of the connector of FIG. 14;

FIG. 17 is a fragmentary, cross-sectional view of another alternative embodiment of the connector according to the invention coupled to a downstream female luer connector;

FIG. 18 is a fragmentary, cross-sectional view of still another alternative embodiment of the connector according to the invention coupled to a downstream female luer connector; and

FIG. 19 is a fragmentary, enlarged cross-sectional view of a portion of the connector of FIG. 18 with a bi-curved flow chamber.

### DESCRIPTION OF THE PREFERRED EMBODIMENTS

As shown in FIG. 3, the different fluids entering the chamber 33 of the connector 32 from input lumen 18, 26, 27 travel towards the catheter 34 and contact the inner distal face 36 of the chamber 33. These fluids, therefore, are forced to interface together on their way into and through the entry orifice 39 of the catheter 34. This uncontrolled mixing is turbulent and transitory and cannot assure a constant and controlled delivery of each of the differing fluids being delivered simultaneously. This is especially true where the fluids have similar viscosities. In some circumstances, gravity could have a pronounced affect on the denser fluids being concomitantly delivered, especially as the diameter of the chamber 33 increases.

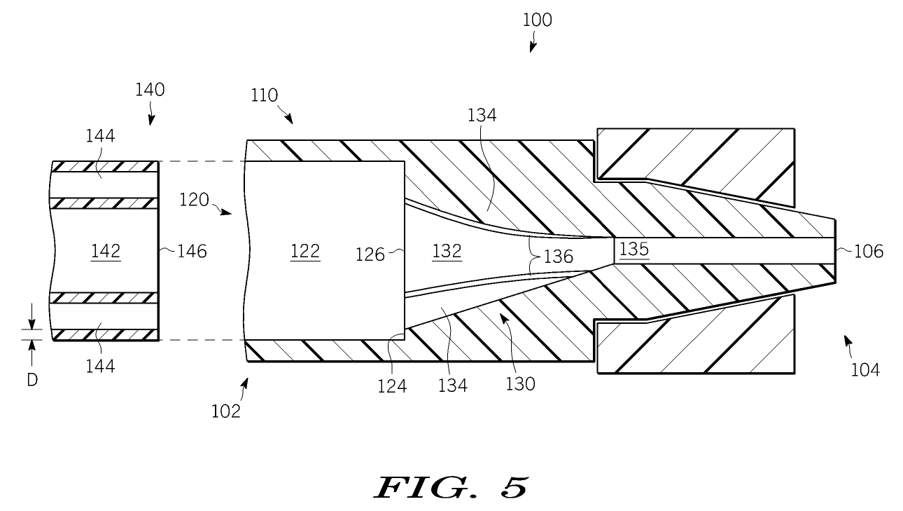

Referring now to the drawings in greater detail, there is illustrated in FIG. 5, a fluid intermixing connector 100 that minimizes or substantially avoids unnecessary or undesired mixing of any combination of the secondary lumen fluids and the primary lumen fluid before being co-delivered, for example, through the intravenous needle/catheter 34 to a patient.

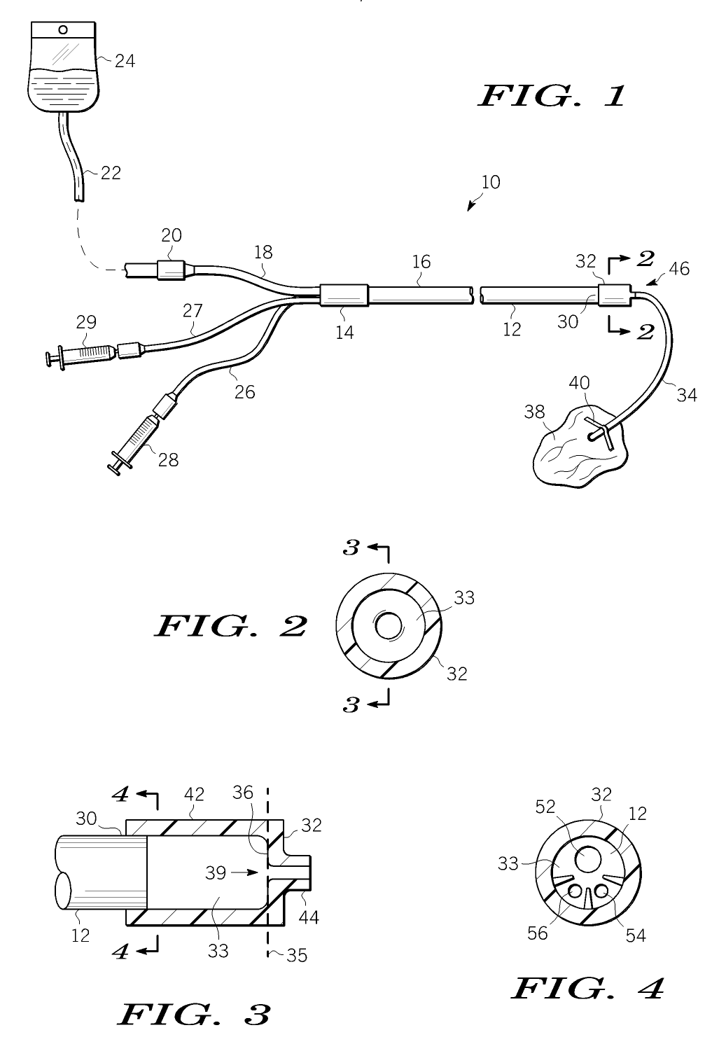

The connector 100 has an outer diameter that can be of a standard size to fit multi-lumen supply lines such as the three-lumen configuration 18, 26, 27 illustrated in FIG. 1. One exemplary size for the outer diameter of the connector 100 has the same outer shape of connector 14. Of course, as long as the fluid supplying lumens can be inserted into the inflow side of the connector 100, the outer diameter of the connector 100 can be any desired size.

The body of the connector 100 defines an interior chamber 110 having two parts, a proximal connection portion 120 and a distal intermixing portion 130.

The proximal connection portion 120 has a substantially cylindrical interior cavity 122 for receiving therein one or more of the fluid supplying lumens, for example, the primary and secondary lumens 18, 26, 27 illustrated in FIG. 1 or the catheters illustrated in U.S. Pat. Nos. 4,968,308 to Dake et al. or 5,833,652 to Preissman et al. If the distal ends of the fluid supplying lumens do not, together, have a cylindrical outer shape, then the interior cavity 122 can be of any shape for receiving these ends. The fluid supplying lumens can be separately inserted into the proximal connection portion 120 or can be bundled into an integral distal end 140 shown, for example, in FIG. 5 and having an outer shape substantially corresponding to the interior shape of the proximal interior cavity 122. In either configuration, these lumens are fluid-tightly fixed to the proximal connection portion 120 within the proximal cavity 122 so that all fluid flow therefrom travels from the input side 102 to the output side 104 of the connector 100.

The interface between the proximal cavity 122 and the distal intermixing portion 130 can include a limiting shelf 124 having an internally projecting radial extent less than or equal to a distance D between the outer circumference of the distal end of the multi-lumen tube assembly and the radially outer-most edge of an opening of any of the lumens within the distal end 140. For example, if there is a distal end 140 with three lumens aligned along a single diameter as shown in FIG. 5 (secondary 144, primary 142, secondary 144), then the shelf 124 can be as thick as the distance D between the outer-most edge of the secondary lumen 142 and the outer edge of the distal end 140. In such a configuration, the outer-mist lumen 144 would not be obstructed in any way by either the shelf 122 or the internal cavity 132 of the distal intermixing portion 130.

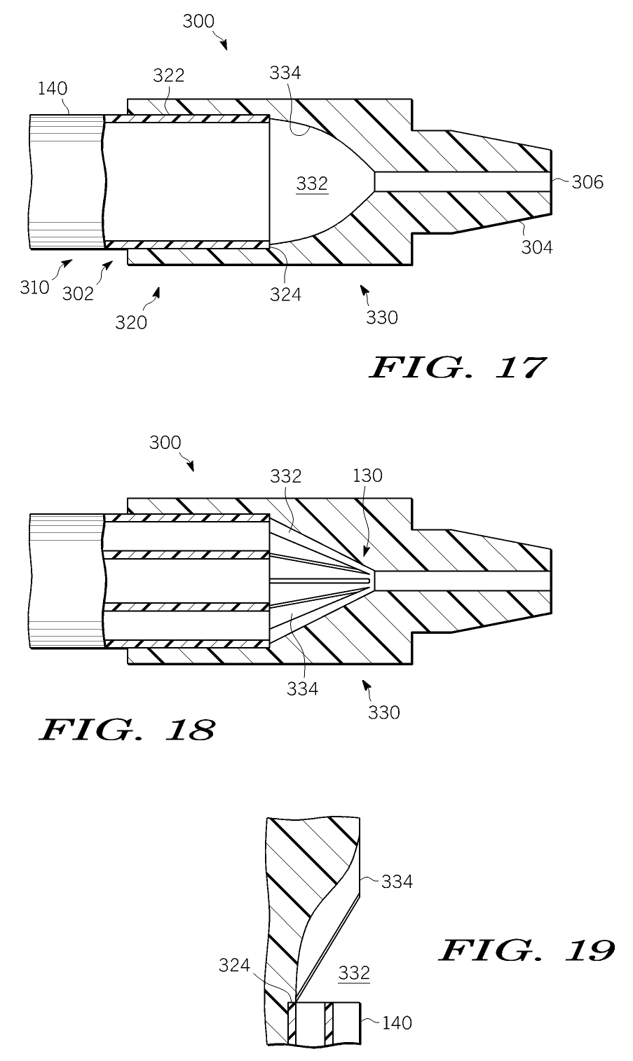

The distal intermixing portion 130 is the downstream portion of the chamber 110. This region receives the fluids that exit the fluid supplying lumens. In one exemplary embodiment illustrated in FIG. 5, the distal intermixing portion 130 is a conical- or funnel-shaped chamber that directs fluid flow from the primary and secondary lumen(s) to the single output bore 106 of the connector 100. In this exemplary embodiment, the distal intermixing portion 130 has a longitudinal length greater than a longitudinal length of the proximal intermixing portion 120. Alternatively, as shown in FIG. 13, the longitudinal length of the distal intermixing portion 130 can be approximately equal to the longitudinal length of the proximal intermixing portion 120 or it can be even shorter than the proximal portion 120, as is illustrated in FIGS. 17 and 18. In cross-section, the cavity 132 can be paraboloid, spherical, or polygonal in its funnel shape. In the latter configuration, the polygonal funnel can have a number of sides equal to, less than, or greater than the number of lumens supplying fluid into the cavity 132. FIG. 17, for example, shows a paraboloidal-shaped funnel.

Consistent with the Coanda Effect, the fluids exiting secondary lumens that are disposed adjacent the inner wall 132 of the funnel will travel along that surface and remain substantially coherent along the inwardly curved/slanted wall 132 with little or no mixing with the primary lumen fluid (or other secondary fluids). This laminar flow is maintained due to the streamlining (described as the Coanda Effect above) that is created by the wall 132 of the proximal intermixing portion 130 from the lumen exit to the bore 106.

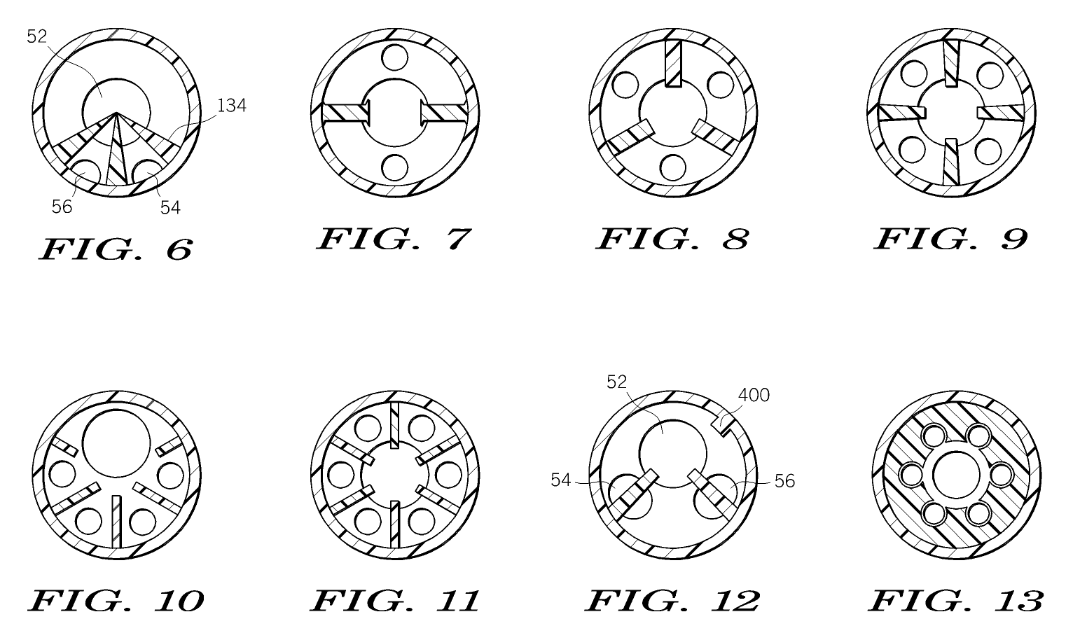

It can be appreciated that laminar flow can be enhanced if guiding fins 134 project inwardly from the inner wall 132. Such fins 134 are illustrated, first, within the distal cavity 132 of the connector 100 illustrated in FIG. 5. By extending radially into the funnel shaped chamber, these fins 134 partition the flows. If the number of fins 134 is equal to the number of fluid supplying lumens, then the distal cavity 132 can be divided into portions that enhance the laminar flow of each fluid being supplied by the lumens. FIG. 6, for example, shows such a configuration. In this exemplary embodiment, the fins have a trapezoidal cross-sectional shape. Of course, any cross-sectional shape can be used, such as the rectangular shape of the fins 134 in FIGS. 10 and 11, the curved I-beam shape of the fins 134 in FIG. 7, or the complex-curved flower-like shape the fins 134 create in FIG. 13.

In the exemplary embodiment of FIG. 5, the fins 134 have a height that is approximately equal to the radial distance between the inner surface of the cavity 132 and the inner-most edge of the secondary lumen 54, 56. These fins 134 can be any height and can even touch at a center point between the three lumens 52, 54, 56 as shown in FIG. 6, for example. The touching/connection of the fins 134 at the center point can occur either only at the distal end of the fins 134 or can extend most of the way to the exit 135 of the distal cavity 132 so that, when viewed from a downstream end of the connector 100, the bore 106 has a pie-chart cross-section as illustrated in FIG. 6.

The fins 134 have differing configurations depending upon the spatial orientation of the primary and secondary lumens. FIGS. 6 to 13 illustrate various exemplary configurations of the fins 134 within the distal cavity 132 numbering fins from 2 to 6. Of course, manufacturing limitations and needs of the user will determine whether or not a given number of fins 134 is practical for the desired use.

The interior edges of the fins 134 can be sharpened with a beveled edge 136 like a knife to improve segregation and decrease turbulence thereat. Such an embodiment is shown, for example, in FIGS. 5 and 19.

Each of the fins 134 in FIGS. 6 to 11 and 13 are illustrated as being disposed to a side of a secondary lumen. If desired, one or more fins 134 can bisect one or more of the secondary lumens. FIG. 12 illustrates a bisection of two secondary lumens 54, 56. Such a configuration may be useful where the viscosity(ies) of one or more of the fluids to be delivered through the connector 100 make the fluids difficult to intermix. If, for example, the first fluid of the primary lumen 52 is significantly less viscous than the second and third fluids exiting from the secondary lumens 54, 56, it may be desirable to “pre-mix” portions of the second and third fluids with the first fluid and, thereby, “increase” the viscosity of the first fluid. In this way, when the mixtures at the distal end of the fins 134 approach the exit 135 of the distal cavity, the less viscous fluid does not “beat out” the other fluids in the “race” through the exit and, thereby, prevent the more viscous fluid(s) from exiting the connector 100.

The shape of the sides of the fins 134 can take many forms, triangular, rectangular, polygonal, blade- or knife-shaped, and/or a combination of one or more shapes. FIG. 5, for example, shows a triangular blade-shaped side in the lower of the two fins 134 and a curvilinear blade-shaped side in the upper of the two fins 134. One particularly well-performing fin configuration is shown in FIG. 13.

In the fins 134 extend all the way to the distal surface 146 of the distal end 140 of the fluid supplying lumens, then the fins 134 can abut the distal surface 146 (as shown at the lower of the two fins 134 of FIG. 5) and entirely replace the limiting shelf 124. In such a configuration, the proximal surfaces of the fins 134 will form the limiting shelf 124 that prevents the distal end 140 from entering the cavity 132 of the distal portion 130. Of course, if the fins 134 extend any part of the way towards the center of the cavity 132 at the interface 126 of the proximal 122 and distal 132 cavities, the proximal surface of the fins 134 lying in a plane transverse to the longitudinal extent of the connector 100 will prevent further movement of the distal end 140 into the cavity 132.

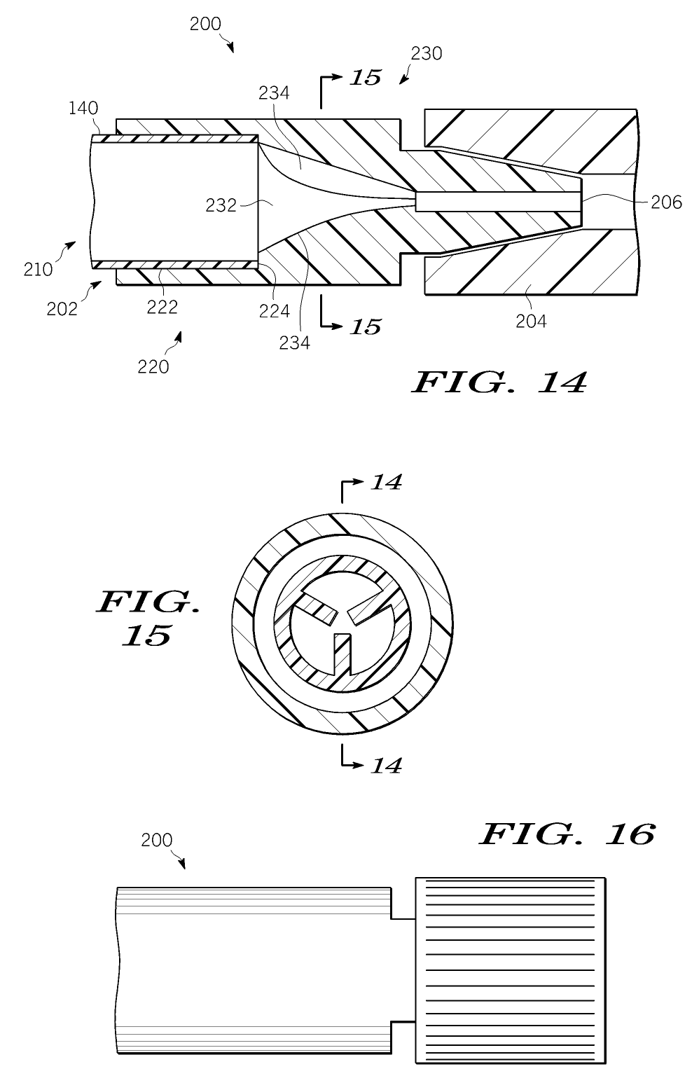

FIGS. 14 to 16 illustrate another alternative embodiment of a connector 200 of the present invention. FIG. 14 is a cross-section through the section line illustrated in FIG. 15. The connector 200 has an interior chamber 210 with two parts, a proximal connection portion 220 and a distal intermixing portion 230. The connector 200 has an input side 202 for receiving the distal end 140 in the proximal cavity 222 and an output side 204 for delivering the intermixed fluids out from the exit bore 206. The configuration of FIGS. 14 to 16 has a distal cavity 232 with a smaller longitudinal extent than the cavity 132 of the connector 100 shown in FIG. 5. The proximal portion 220 has a cylindrical proximal cavity 222 with a distal stopping shelf 224 that prevents distal insertion of the distal end 140 into the distal cavity 232. Of course, the fins 234 can provide the distal stopping shelf on their upstream side.

FIG. 17 illustrates a further alternative embodiment of a connector 300 of the present invention. The connector 300 has an interior chamber 310 having two parts, a proximal connection portion 320 and a distal intermixing portion 330. The connector 300 has an input side 302 for receiving the distal end 140 in the proximal cavity 322 and an output side 304 for delivering the intermixed fluids out from the exit bore 306. In this cross-section, the distal cavity 332 has a smaller longitudinal extent than the cavity 132 of the connector 100 shown in FIG. 5. The distal cavity 332 does not have fins and is funnel shaped with a linear inwardly sloping wall 334. The proximal portion 320 has a cylindrical proximal cavity 322 with a distal stopping shelf 324 that prevents distal insertion of the distal end 140 into the distal cavity 332.

FIG. 18 is the connector 300 of FIG. 17 but with eight fins 334 spaced evenly about a non-curvilinear funnel-shaped cavity 332. FIG. 19 is an enlarged portion of one of the fins 334 and illustrates a height of the fins 334 that is increasing from the proximal side of the distal cavity 332 towards the distal side thereof. This fin 334 also illustrates a distal cavity 332 that is paraboloidal concave with a convex exit to create a smooth transition at the exit of the cavity 332.

The desired orientation of the multi-lumens with respect to the fins 134, 234, 334, may require exact placement of the distal end 140 of the multiple lumens. Exact rotational orientation can be assured by providing at least one recess on the exterior surface of the distal end 140 of the multi-lumen plug that is to be inserted into the proximal cavity 132, 232, 332 of the connector 100, 200, 300. If the proximal cavity 132, 232, 332 is provided with at least one protrusion extending radially inward into the center of the cavity 132, 232, 332, then the distal end 140 of the lumens to be inserted therein will not occur unless the protrusion is aligned with the recess—much like a key and keyhole. Of course, this configuration can be reversed if desired. If only one recess and only one protrusion is provided according to such a configuration, then the distal end 140 cannot enter the proximal cavity 132, 232, 332 except in proper rotational alignment. An example of this single recess/protrusion assembly 400 is illustrated in the cross-section of FIG. 12. When there exist more than one accepted rotational orientation of the distal end, such as the symmetric configurations of FIGS. 7, 8, 9, and 11, it is possible to include more than one recess/protrusion. For example, the configuration of FIG. 7 can have two symmetrical recesses/protrusions, the configuration of FIG. 8 can have three symmetrical recesses/protrusions, the configuration of FIG. 9 can have four symmetrical recesses/protrusions, and the configuration of FIG. 11 can have six symmetrical recesses/protrusions.

The protrusion on the inside of the chamber 132, 232, 332 can be displayed to the user, if desired, in directions for use or can be permanently marked on the connector 100, 200, 300.

There are many kinds of luer connector fittings that can be used with the connector 100, 200, 300. Only a few exemplary embodiments are illustrated in the figures of the drawings and, therefore, the possible luer fittings should not be limited to that which is shown. The fittings typically include round male and female interlocking tubes, slightly tapered to hold together better with a simple pressure or twist fit, referred to in the art as a luer slip and a luer lock. In the latter configuration, an outer threading rim improves the secure, fluid-tight connection of the luer connector.

One advantage to each of the above-mentioned configurations over the '167 device is that the volume of the intermixing chamber 132, 232, 332 is smaller than the pill-shaped chamber 32. Therefore, the amount of medicinal fluid necessary to fill the chamber 132, 232, 332 is reduced, thereby, decreasing the time for any injectate to exit the connector and enter the catheter 34. Also the volume of priming/flushing fluids is reduced as well as the time taken to prime or flush.

The connector 100 of the present invention can be used in a number of medical applications. For example, it can be used in anesthesia during operations for infusion of anesthetic agents, vaso-active agents, antibiotics, and antiarrhymics, whether in adults or children (both neonatal and pediatric). The connector 100, 200, 300 also can be used with a PCA pump and can be used in an intensive care unit for vaso-active meds, antiarrhymics, potassium, antibiotics, insulin, etc.

The '167 Patent describes an over-pressure danger that exists at a vascular entry point when fluid is being introduced into a patient. As can be understood from the description of the connector 100, 200, 300, replacement of the '167 connector 32 with the connector 100, 200, 300 does not adversely impact the over-pressure protection that exists when the connector 100, 200, 300 is utilized with the '167 system 10 and, therefore, is particularly suited for improving that system 10.

Because turbulence of the intermixed fluids at the connector 100, 200, 300 is minimized, all of the advantages provided by the '167 system remain with the connector 100, 200, 300. More specifically, delivering the pharmaceutical agents with fewer changes to the normal fluid dynamics improves patient safety as compared to prior art infusion systems. Additionally, the infusion of liquid agents through the satellite lumens remains independent of carrier fluid rates for delivery. Because the liquids from the satellite lumens are delivered with greater control in volume, the time of the onset of the action of the agents delivered is decreased and the concentration of those agents remains virtually constant. Less intermixing of the fluids also means that delivery of the agents infused through the satellite lumens will not be altered by the carrier fluid rate. Like the '167 system, the connector 100, 200, 300 of the present invention decreases priming volume even more by further reducing the “tubing dead space.” The connector 100, 200, 300 also allows and enhances independent infusion of multiple agents and reduces carrier fluid rate requirements.

From the foregoing description, it will be appreciated that the connector of the present invention provides a number of advantages, some of which have been described above and others of which are inherent in the invention.

I claim:

1. A fluid turbulence minimizing device comprising:
a connection portion defining a substantially cylindrical connection cavity shaped to receive a cylindrical fluid-supply conduit including a primary fluid-supply lumen and at least one secondary fluid-supply lumen; and
an intermixing portion physically coupled to the connection portion, the intermixing portion defining an intermixing cavity with:
an inner surface having a first diameter at a side proximate to the connection portion and a second diameter distal from the connection portion, the first diameter greater than the second diameter;
an exit orifice, the intermixing cavity fluidically communicating with the connection cavity to convey fluid supplied to the connection cavity through the intermixing cavity and out from the exit orifice; and
at least one guide fin inwardly projecting from the inner surface of the intermixing portion toward a center thereof.

2. The fluid turbulence minimizing device according to claim 1, wherein the at least one guide fin has a longitudinally cross-sectional shape selected from at least one of the group consisting of:
a triangle;
a rectangle;
a polygon;
a blade;
an I-beam; and
a curved I-beam.

3. The fluid turbulence minimizing device according to claim 1, wherein the at least one guide fin is at least three fins together having a radially cross-sectional flower shape.

4. The fluid turbulence minimizing device according to claim 1, wherein a quantity of the at least one guide fins is equal to a quantity of the lumens in the conduit.

5. The fluid turbulence minimizing device according to claim 1, wherein the at least one guide fin is disposed at a location approximately half way between two adjacent lumens when the conduit is placed within the connection cavity.

6. The fluid turbulence minimizing device according to claim 1, wherein the at least one guide fin is disposed at a location substantially aligned with a center of at least one of the primary fluid-supply lumen and the at least one secondary fluid-supply lumen when the conduit is placed within the connection cavity.

7. The fluid turbulence minimizing device according to claim 1, wherein a shape of the inner surface of the intermixing portion is substantially a funnel.

8. The fluid turbulence minimizing device according to claim 1, wherein the funnel shape is at least one of:
paraboloid;
spherical; and
polygonal.

9. The fluid turbulence minimizing device according to claim 1, further comprising:
a wall located at a junction between the connection portion and the intermixing portion, the wall extending substantially perpendicularly from the inner surface of the intermixing portion to a height approximately equal to a distance from an outer surface of the conduit to a nearest interior surface of the secondary fluid-supply lumen.

10. A fluid turbulence minimizing device comprising:
a connection portion defining a connection cavity shaped to receive a tertiary fluid-supply conduit including a primary fluid-supply lumen and two secondary fluid-supply lumens; and
an intermixing portion coupled to the connection portion, having a longitudinal flow axis, and defining an intermixing cavity with:
an input orifice fluidically communicating with the connection cavity and having a given area;
an exit orifice having an area less than the given area;
an inner surface having an upstream side adjacent the connection portion, a downstream side at a distance from the connection portion, and a cross-sectional area decreasing from the input orifice to the exit orifice to convey fluid supplied from the conduit to the connection cavity through the intermixing cavity and out the exit orifice; and
at least one guide fin inwardly projecting from the inner surface of the intermixing portion toward the longitudinal flow axis and having a longitudinal extent aligned substantially parallel with the longitudinal flow axis.

11. The fluid turbulence minimizing device according to claim 10, wherein
the connection portion, the intermixing portion, and the at least one guide fin are integral.

12. The fluid turbulence minimizing device according to claim 10, wherein
the connection cavity is substantially cylindrical for receiving a substantially cylindrical conduit.

13. The fluid turbulence minimizing device according to claim 10, wherein a shape of the inner surface decreases at least one of linearly, exponentially, geometrically, in steps, and gradually from the input orifice to the exit orifice.

14. The fluid turbulence minimizing device according to claim 10, wherein the at least one guide fin has a longitudinally cross-sectional shape selected from at least one of the group consisting of:
a triangle
a rectangle;
a polygon;
a blade;
an I-beam; and
a curved I-beam.

15. The fluid turbulence minimizing device according to claim 10, wherein the at least one guide fin is at least three fins together having a radial cross-section in the shape of a flower.

16. A method of reducing fluid turbulence when transferring fluid from multiple lumens to a single conduit, the method comprising:
receiving, at a connection portion, fluid from a fluid-supply conduit that includes a primary fluid-supply lumen and at least one secondary fluid-supply lumen;
conveying the fluid from the connection portion into an intermixing cavity that has:
an inner surface with a first diameter at a side proximate to the connection portion and a second diameter distal from the connection portion, the first diameter greater than the second diameter;
an exit orifice, the intermixing cavity fluidically communicating with the connection cavity to convey fluid supplied to the connection cavity through the intermixing cavity and out from the exit orifice; and
at least one guide fin inwardly projecting from the inner surface of the intermixing portion toward a center thereof; and
discharging the fluid from the exit orifice.

17. The method according to claim 16, wherein the at least one guide fin has a longitudinally cross-sectional shape selected from at least one of the group consisting of:
a triangle;
a rectangle;
a polygon;
a blade;
an I beam; and
a curved I-beam.

18. The method according to claim 16, wherein the at least one guide fin is at least three fins together having a radially cross-sectional flower shape.

19. The method according to claim 16, wherein a quantity of the at least one guide fins is equal to a quantity of the lumens in the conduit.

20. The method according to claim 16, which further comprises disposing the at least one guide fin at a location approximately half way between the primary fluid-supply lumen and the at least one secondary fluid-supply lumen when the conduit is placed within the connection cavity.

21. The method according to claim 16, which further comprises disposing at least one guide fin at a location substantially aligned with a center of at least one of the lumens when the conduit is placed within the connection cavity.

22. The method according to claim 16, wherein a shape of the inner surface of the intermixing portion is substantially a funnel.

23. The method according to claim 22, wherein the funnel shape is at least one of:
paraboloid;
spherical; and
polygonal.

24. The method according to claim 16, wherein:
the connection portion and the intermixing portion are separated by a wall located at a junction between the connection portion and the intermixing portion, the wall extending substantially perpendicularly from the inner surface of the intermixing portion to a height approximately equal to a distance from an outer surface of the conduit to a nearest interior surface of the secondary fluid-supply lumen.

25. A method of reducing fluid turbulence when transferring fluid from multiple lumens to a single conduit, the method comprising:
Providing an intermixing cavity with:
an entrance orifice having a given area;
an exit orifice having an area smaller than the given area;
an inner conduit surface fluidically connecting the entrance and exit orifices and decreasing in size from the entrance orifice to the exit orifice; and
at least one guide fin inwardly projecting from the inner conduit surface toward a center thereof;
fluidically connecting the intermixing cavity with a connection portion for transmitting fluid supplied from the connection portion through the intermixing cavity;
receiving, at the connection portion, a first fluid from a primary fluid-supply lumen and a second fluid from at least one secondary fluid-supply lumen of a multi-lumen fluid-supply conduit;
intermixing the first and second fluids in the intermixing cavity while retaining fluid separation with the at least one guide fin; and
dispensing the intermixed first and second fluids out from the exit orifice.
